(19) United States
(12) Patent Application Publication
(10) Pub. No.: US 2019/0015632 A1
(43) Pub. Date: Jan. 17, 2019

(54) MEDICINE INJECTING CATHETER AND MEDICINE INJECTING SYSTEM

(71) Applicant: TERUMO KABUSHIKI KAISHA, Tokyo (JP)

(72) Inventors: Naoya Shimada, Kanagawa (JP); Naoki Ishii, Kanagawa (JP)

(73) Assignee: TERUMO KABUSHIKI KAISHA, Tokyo (JP)

(21) Appl. No.: 16/126,595

(22) Filed: Sep. 10, 2018

Related U.S. Application Data
(63) Continuation of application No. PCT/JP2017/009662, filed on Mar. 10, 2017.

(30) Foreign Application Priority Data
Mar. 11, 2016 (JP) 2016-047972

(51) Int. Cl. A61M 25/00; A61M 25/04; A61M 25/01

(52) U.S. Cl. CPC A61M 25/0084; A61M 25/04; A61M 2025/0089; A61M 2205/3344; A61M 2210/125; A61M 25/0108

(57) ABSTRACT

A medicine injecting catheter includes a catheter body having a medicine supply lumen, a tubular body coupled to a distal end of the catheter body and having a lumen fluidly communicating with the medicine supply lumen, an injection needle connected to a distal end of the tubular body, a cover member having an interior housing the tubular body, with the cover member disposed on the distal end of the catheter body for reciprocating axial movement between a first position in which the distal end of the needle is in the inner space and a second position in which the distal end of the needle projects forwardly from the inner space, and an opening/closing mechanism for opening/closing the tubular body lumen depending on an angular position of an operating member, and a motion converting mechanism for converting reciprocating movement of the cover member into rotary movement of the operating member.

### CROSS-REFERENCES TO RELATED APPLICATIONS

This application is a continuation of International Application No. PCT/JP2017/009662 filed on Mar. 10, 2017, which claims priority to Japanese Patent Application No. 2016-047972 filed on Mar. 11, 2016, the entire content of both of which is incorporated herein by reference.

### TECHNICAL FIELD

The present invention generally relates to a medicine or medication injecting catheter, and more particularly a medicine or medication injecting catheter for injecting medication or medicine into a living body through a body wall of the living body. The present invention is also concerned with a medication or medicine injecting system that employs such a medication or medicine injecting catheter.

### BACKGROUND DISCUSSION

When the pumping function of a heart fails due to any of various causes, the heart is unable to pump enough blood required for tissue metabolism in the whole body. As one therapeutic method for such a heart failure, there has been studied a noteworthy process of injecting a medicine made of biomaterials such as growth factors, genes, or cells, etc. into a damaged region such as a myocardial infarct.

Such a medicine may be injected into the heart by way of a thoracotomy. To make the injecting process less invasive, however, Japanese Application No. T-2004-528062, for example, discloses a medical instrument having a puncture unit provided on the distal end of a catheter. The puncture unit is used to puncture a body wall such as a cardiac muscle or the like with a needle-like device thereof and to inject the medicine through the needle-like device into the body wall.

### SUMMARY

However, the disclosed medical instrument is problematic in that the procedure for sticking the needle-like device into the body wall is complex and time-consuming.

Furthermore, since it is not possible to confirm whether the needle-like device has pierced the body wall or not, the medicine may possibly leak out of the needle-like device around the body wall when approximately the needle-like device pierces the body wall. If the medicine leaks around the body wall, then there is a possibility that it may cause a new disease or it may be difficult to administer a proper amount of medicine to the target region.

The medicine injecting catheter disclosed here is capable of injecting a medicine reliably into a living body from a body wall of the living body without leaking the medicine in a simple procedure.

Also disclosed is a medicine injecting system that employs such a medicine injecting catheter.

A medicine injecting catheter for injecting medication into a wall of a living body comprises: an elongate catheter body extending in the axial direction, with the catheter body including a medicine supply lumen extending in the catheter body, a tubular body connected to the distal end of the catheter body, with the tubular body including a lumen in fluid communication with the medicine supply lumen; an injection needle connected to the distal end of the tubular body; a hollow cover member that includes an inner space in which is accommodated at least the tubular body, with the cover member being disposed on a distal end portion of the catheter body for reciprocating movement along the axial direction of the catheter body between a first position in which the distal end of the injection needle is housed in the inner space and a second position in which the distal end of the injection needle projects forwardly from the inner space; and an opening and closing mechanism that includes an operating member rotatable in opposite circumferential directions of the catheter body in the inner space in the cover member, for opening and closing the lumen in the tubular body depending on an angular position of the operating member. A motion converting mechanism converts the reciprocating movement of the cover member relative to the catheter body into rotary movement of the operating member to cause the opening and closing mechanism to close the lumen in the tubular body when the cover member is positioned in the first position and to cause the opening and closing mechanism to open the lumen in the tubular body when the cover member is positioned in the second position. A resilient member disposed between the catheter body and the cover member and resiliently presses the cover member forwardly of the catheter body toward the first position.

The tubular member may be pliable, and the opening and closing mechanism may include a diaphragm mechanism for changing the opening area of the lumen in the tubular body depending on the angular position of the operating member. Alternatively, the tubular member may be pliable, and the opening and closing mechanism may include at least one rotor for twisting the tubular body depending on the angular position of the operating member.

The motion converting mechanism should preferably have an operating member guide groove defined helically in an inner circumferential surface of the cover member around a central axis of the cover member, and the operating member should preferably include a projection extending radially of the catheter body in the inner space in the cover member and movably inserted in the operating member guide groove.

Preferably, the cover member should have a partition plate fixedly disposed in the inner space thereof perpendicularly to the axial directions of the catheter body and having a through hole defined therein with the injection needle extending therethrough, and the resilient member should be disposed between a front end of the catheter body and the partition plate of the cover member.

The medicine injecting may further include a capture mechanism for capturing a body wall surface of a living body into a front end portion of the cover member by evacuating the inner space in the cover member.

The catheter body and the cover member should preferably have respective contrast markers. The contrast markers make it possible to confirm, from outside, the manner in which the cover member moves and the injection needle pierces a body wall such as a heart muscle or the like. In addition, the practitioner is preferably given information regarding how the catheter body, particularly the distal end thereof, is twisted and the direction in which the catheter body is oriented, depending on how the pattern of the contrast markers appears.

A medicine injecting system according to the present invention includes the above medicine injecting catheter, a pressure gage for detecting a pressure in the medicine supply lumen of the medicine injecting catheter, a medicine supply for supplying a medicine to the medicine supply lumen of the medicine injecting catheter, and a controller for controlling the supplying of the medicine from the medicine supply so that the pressure detected by the pressure gage in the medicine supply lumen of the medicine injecting catheter is constant.

According to the disclosed medicine injecting catheter, the cover member with the inner space defined therein is reciprocally movable between the first position in which the distal end of the injection needle is housed in the inner space and the second position in which the distal end of the injection needle projects forwardly from the inner space, the motion converting mechanism converts reciprocating movement of the cover member with respect to the catheter body into rotary movement of the operating member, and the opening and closing mechanism opens and closes the lumen in the tubular body depending on the angular position of the operating member, to close the lumen in the tubular body when the cover member is positioned in the first position and to open the lumen in the tubular body when the cover member is positioned in the second position. It is thus possible to inject the medicine reliably into the living body through the body wall thereof without leaking the medicine in a simple procedure.

According to another aspect, a medicine injecting catheter for injecting medicine into a wall of a living body comprises: an elongate catheter body extending in an axial direction, with the catheter body including a medicine supply lumen extending in the axial direction inside the catheter body and opening to the distal end of the catheter body; a tubular body connected to the distal end of the catheter body, with the tubular body including a lumen and being made of compressible material that is compressed upon application of a compressive force to the outer periphery of the tubular body to close the lumen in the tubular body; an injection needle connected to the tubular body and including a lumen that opens to the distal end of the injection needle; wherein the catheter body, the tubular body and the injection needle are movable together as a unit; and a cover member surrounding an interior in which is located at least a portion of the tubular body, with the cover member and the catheter body being relatively movable between a first position in which a distal-most end of the injection needle is proximal of a distal-most end of the cover member so that the distal-most end of the injection needle is covered by the cover member and a second position in which the distal-most end of the injection needle protrudes distally beyond the distal-most end of the cover member so that the distal-most end of the injection needle is outside the tubular cover to puncture the living body. An opening and closing mechanism closes the lumen in the tubular body and opens the lumen in the tubular body. The opening and closing mechanism comprises at least one tubular body contacting part movable between one position in which the tubular body contacting part contacts the tubular body and applies the compressive force to the tubular body that closes the lumen in the tubular body and an other position in which the tubular body contacting part no longer applies the compressive force to the tubular body so that the lumen in the tubular body is open. The opening and closing mechanism also comprises an operating member that is operatively connected to the tubular body contacting part and the cover member so that when the cover member is in the first position with the distal-most end of the injection needle covered by the cover member, the tubular body contacting part is in the one position to close the lumen in the tubular body and prevent medicine from flowing into the injection needle, and when the cover member is shifted to the second position with the distal-most end of the injection needle protruding distally beyond the distal-most end of the cover member, the tubular body contacting part is moved to the other position so that the lumen in the tubular body is open to permit medicine to flow into the injection needle.

In accordance with another aspect, a method comprises: inserting a catheter body into a patient's body, wherein the catheter body includes a medicine supply lumen extending in the catheter body and opening at the distal end of the catheter body, with the catheter body being connected to a tubular body which includes a lumen in communication with the medicine supply lumen, with the tubular body being connected to an injection needle which possesses a distal end, with the catheter body and the injection needle being movable together, with at least a portion of the tubular body being positioned in an interior of a cover member, and a rotatable operation member positioned inside the cover member. The method also involves moving the cover member and the catheter body in a forward direction toward a wall inside the patient's body while a compressive force is applied to the tubular body to close the lumen in the tubular body to completely block flow of medicine through the lumen in the tubular body, moving the cover member in the forward direction so that the distal end of the cover member contacts the wall inside the patient's body to stop the movement of the cover member in the forward direction, and moving the catheter body in the forward direction relative to the cover member after the distal end of the cover member contacts the wall inside the patient's body so that the distal end of the injection needle extends distally beyond a distal end of the cover member and punctures the wall of the patient's body. The moving of the catheter body in the forward direction relative to the cover member after the distal end of the cover member contacts the wall inside the patient's body causing the operation member to rotate, and the rotation of the operation member automatically removing the compressive force applied to the tubular body to open the lumen in the tubular body. Following the opening of the lumen in the tubular body, the medicine flows through the medicine supply lumen, through the lumen in the tubular body and through the injection needle so that the medicine exits the injection needle by way of the distal end of the injection needle and is introduced into tissue in the wall of the patient's body.

### BRIEF DESCRIPTION OF THE DRAWING FIGURES

FIG. 1 is a cross-sectional view of a medicine injecting catheter according to Embodiment 1, with a cover member positioned in a first position.

FIG. 2 is a perspective view of a diaphragm mechanism used in the medicine injecting catheter according to Embodiment 1, the diaphragm mechanism being depicted as being closed.

FIG. 3 is a cross-sectional view taken along the section line II-II of FIG. 1.

FIG. 4 is a cross-sectional view of the medicine injecting catheter according to Embodiment 1, with the cover member positioned in a second position.

FIG. 5 is a perspective view of the diaphragm mechanism used in the medicine injecting catheter according to Embodiment 1, the diaphragm mechanism being depicted as being open.

FIG. 6 is a block diagram depicting the makeup of a medical injection system that uses the medicine injecting catheter according to Embodiment 1.

FIG. 7 is a view depicting the medicine injecting catheter according to Embodiment 1, with the cover member disposed in the vicinity of the surface of a body wall.

FIG. 8 is a view depicting the medicine injecting catheter according to

[symbol]

FIG. 9 is a cross-sectional view of a medicine injecting catheter according to Embodiment 2, with a cover member positioned in a first position.

FIG. 10 is a perspective view of a tubular body of the medicine injecting catheter according to Embodiment 2, with the cover member positioned in the first position.

FIG. 11 is a cross-sectional view of the medicine injecting catheter according to Embodiment 2, with the cover member positioned in a second position.

FIG. 12 is a perspective view of the tubular body of the medicine injecting catheter according to Embodiment 2, with the cover member positioned in the second position.

FIG. 13 is a cross-sectional view of a medicine injecting catheter according to Embodiment 3, with a cover member positioned in a first position.

FIG. 14 is a cross-sectional view of the medicine injecting catheter according to Embodiment 3, with the cover member positioned in a second position.

FIG. 15 is a perspective view of a distal end portion of a medicine injecting catheter according to Embodiment 4.

FIG. 16 is a perspective view of a medicine injecting catheter according to Embodiment 5, with a cover member positioned in a first position.

FIG. 17 is a perspective view of the medicine injecting catheter according to Embodiment 5, with the cover member positioned in a second position.

### DETAILED DESCRIPTION

Set forth below with reference to the accompanying drawings is a detailed description of embodiments of a medicine injecting catheter and a medicine injecting system, representing examples of the inventive medicine injecting catheter and a medicine injecting system disclosed here.

### Embodiment 1

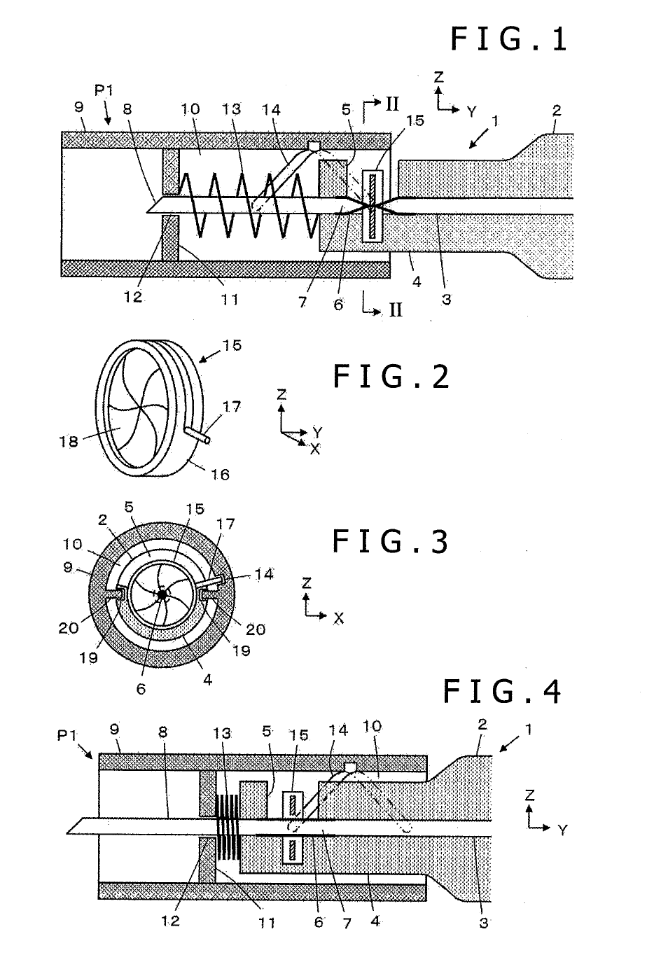

FIG. 1 depicts the makeup of a medicine injecting catheter 1 according to Embodiment 1. The medicine injecting catheter 1 includes an elongate catheter body 2 having a medicine supply lumen 3 defined therein that extends along the axial direction of the catheter body 2.

The catheter body 2 includes a reduced-diameter portion 4 on its distal end portion, which is smaller in diameter than the rest of the catheter body 2. The reduced-diameter portion 4 has a recess 5 defined therein perpendicularly to the axial direction of the catheter body 2. The recess 5 divides or separates the medicine supply lumen 3, with a tubular body 6 disposed across the divided region of the medicine supply lumen 3. The tubular body 6 thus connects or extends between the portion of the medicine supply lumen 3 on the proximal side of the recess 5 and the portion of the medicine supply lumen 3 on the distal side of the recess 5.

The tubular body 6 includes a pliable (compressible) soft tube and has open ends through which a lumen 7 in the tubular body 6 is in fluid communication with the medicine supply lumen 3, thereby connecting the divided ends of the medicine supply lumen 3. An injection needle 8 is held on the distal end portion of the catheter body 2, and is coupled or connected to the tubular body 6 either directly or through the medicine supply lumen 3.

For illustrative purposes, the axial direction of the catheter body 2 will be referred to as a Y direction, the direction in which the recess 5 is defined in the reduced-diameter portion 4 as a +Z direction (i.e., the positive Z direction or the upward Z direction in FIGS. 1 and 4 for example), and a direction perpendicular to a YZ plane as an X direction.

A hollow cylindrical cover member 9 having a central axis coaxial with the catheter body 2 is disposed on the distal end portion of the catheter body 2 in surrounding relation to the reduced-diameter portion 4. The cover member 9 has an inner space or interior 10 that houses the reduced-diameter portion 4, and may have a partition plate 11 disposed in the inner space in the vicinity of the distal end of the cover member 9 and extending in an XZ plane, dividing the inner space 10 into two areas in the Y direction. The partition plate 11 has a through hole 12 defined centrally in the partition plate 11 and extending in the Y direction. The through hole 12 has a diameter larger than the outside diameter of the injection needle 8, which extends through the through hole 12.

A resilient member 13 which may be in the form of a helical spring is disposed between the partition plate 11 in the cover member 9 and the front end of the catheter body 2, and the injection needle 8 extends through the resilient member 13. The resilient member 13 is not limited to a spring, but may be any member that is resilient.

The cover member 9 has an operating member guide groove 14 defined in an inner wall surface of the cover member. The operating member guide groove 14 is disposed only in the inner wall surface of the cover member 9 that lies in a +Z direction, of the entire inner wall surface of the cover member 9, in agreement with the direction along which the recess 5 is defined in the catheter body 2. The operating member guide groove 14 being disposed only in the +Z direction is depicted in FIGS. 1 and 4 for example. The operating member guide groove 14 extends helically around the central axis of the cover member 9 along the Y direction.

A diaphragm mechanism (diaphragm) 15 for changing the opening area of the lumen 7 in the tubular body 6 is disposed in the recess 5 in the catheter body 2. The diaphragm mechanism 15 serves as an opening and closing mechanism for changing the opening area of the lumen 7 in the tubular body 6 to open and close the lumen 7 in the tubular body 6 by pressing an outer circumferential portion (outer peripheral surface) of the tubular body 6 all around the tubular body 6 toward the central axis of the tubular body 6. As depicted in FIG. 2, the diaphragm mechanism 15 has a hollow cylindrical frame 16, an operating member 17 extending radially outwardly from the frame 16, and a plurality of blades 18 arrayed circumferentially inside the frame 16. As shown in FIGS. 1 and 4, the tubular body 6 passes through the diaphragm mechanism 15/frame 16. The operating member 17 is movable along an outer circumferential portion of the frame 16 in circumferential directions of the frame 16. The blades 18 are arranged to be opened and closed in an XZ plane depending on the angular position of the operating member 17.

The operating member 17 has a distal end positioned in the operating member guide groove 14 defined in the inner wall surface of the cover member 9, so that the angular position of the operating member 17 varies depending on the relative position of the cover member 9 with respect to the catheter body 2 in the Y direction. The operating member guide groove 14 of the cover member 9 serves as a motion converting mechanism.

As depicted in FIG. 3, the reduced-diameter portion 4 of the catheter body 2 has a pair of cover member guide grooves 19 defined in an outer circumferential surface of the reduced-diameter portion 4 of the catheter body 2 and extending in the Y direction at the end of the reduced-diameter portion 4 in a +X (i.e., the positive X direction or the rightward X direction in FIG. 3 for example) direction and the end of the reduced-diameter portion 4 in a −X direction (i.e., the negative X direction or the leftward X direction in FIG. 3 for example). The cover member 9 has a pair of respective projections 20 disposed on the end of an inner surface of the cover member 9 in the +X direction and the end of an inner surface of the cover member 9 in the −X direction, the projections 20 projecting into the inner space 10 in the cover member 9 and extending in the Y direction.

The protrusions 20 of the cover member 9 are inserted or positioned in the respective cover member guide grooves 19 of the reduced-diameter portion 4, so that the cover member 9 is held on the catheter body 2 for reciprocating movement along the axial directions of the catheter body 2 between a first position P1 in which the distal end of the injection needle 8 in a −Y direction (i.e., the negative Y direction or the leftward Y direction in FIGS. 1 and 4 for example) is housed in the inner space 10 as depicted in FIG. 1 and a second position P2 in which the distal end of the injection needle 8 in the −Y direction projects forwardly, i.e., in the −Y direction as depicted in FIG. 4.

When the cover member 9 is positioned in the first position P1 depicted in FIG. 1, the operating member 17 of the diaphragm mechanism 15 that is inserted in the operating member guide groove 14 of the cover member 9 is in an angular position oriented essentially in the +X direction as depicted in FIG. 3, in which the blades 18 of the diaphragm mechanism 15 are closed, squeezing the outer circumferential portion of the tubular member 6 disposed in the opening 5 in the catheter body 2 thereby to close the lumen 7 in the tubular body 6 which is in the form of a soft tube. Thus, as shown in FIG. 4, the blades 18 are a tubular body contacting part that contacts the tubular body 6 and applies an inwardly directed compressive force on the tubular body 6 that closes the lumen 7 in the tubular body 6.

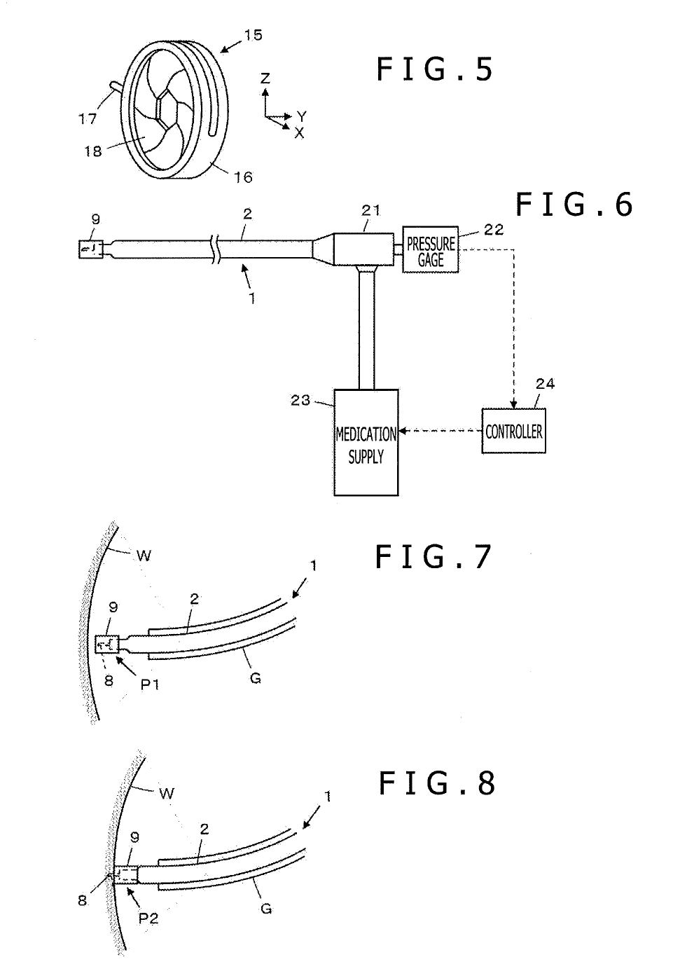

When the cover member 9 moves in a +Y direction (i.e., the positive Y direction or the rightward Y direction in FIGS. 1 and 4 for example) until it is positioned in the second position P2 depicted in FIG. 4 with respect to the catheter body 2, since the operating member guide groove 14 of the cover member 9 extends helically, the operating member 17 of the diaphragm mechanism 15 that is inserted in the operating member guide groove 14 of the cover member 9 is brought into an angular position oriented essentially in the −X direction, opening the blades 18 of the diaphragm mechanism 15 thereby to open the lumen 7 in the tubular body 6, as depicted in FIG. 5.

In other words, when the cover member 9 is positioned in the first position P1 in which the distal end of the injection needle 8 in the −Y direction is stored or housed in the inner space 10, the lumen 7 in the tubular body 6 is closed, and when the cover member 9 is positioned in the second position P2 in which the distal end of the injection needle 8 in the −Y direction projects forwardly from the inner space 10, the lumen 7 in the tubular body 6 is open.

In addition, the cover member 9 is resiliently pressed forwardly with respect to the catheter body 2, i.e., in the −Y direction, by the helical resilient member 13 that is disposed between the partition plate 11 in the cover member 9 and the front end of the catheter body 2. Therefore, unless an external force acts on the cover member 9, the cover member 9 is positioned in the first position P1 depicted in FIG. 1.

The catheter body 2 should preferably be made of a material which is flexible to a certain extent, such as metal or resin. The metal may be, for example, pseudoelastic alloy (including superelastic alloy) such as Ni—Ti alloy, stainless steel (for example, all SUS types such as SUS304, SUS303, SUS316, SUS316L, SUS316J1, SUS316J1L, SUS405, SUS430, SUS434, SUS444, SUS429, SUS430F, SUS302, etc.), cobalt alloy, precious metal such as gold or platinum, tungsten-base alloy, carbon-base material (including piano wire), or the like. The resin may be, for example, polyolefin (for example, polyethylene, polypropylene, polybutene, ethylene-propylene copolymer, ethylene-vinyl acetate copolymer, ionomer, a mixture of two or more of these materials, or the like), a polymeric material such as polyvinyl chloride, polyamide, polyamide elastomer, polyester, polyester elastomer, polyurethane, polyurethane elastomer, polyimide, fluororesin, or the like, or a mixture of these materials, or two or more of the above polymeric materials. The resin may also be engineering plastics typified by polyetheretherketone. The catheter body 2 may also include a multilayer tube made of a composite material including any of the metal and resin materials referred to above.

The cover member 9 may also be made of the similar material as the catheter body 2.

The tubular body 6 may be made of a pliable material such as silicone rubber or the like, for example.

The operating member 17 and the blades 18 of the diaphragm mechanism 15 may be made of a sufficiently rigid material, such as a metal material or a resin material, for example.

FIG. 6 depicts the makeup of a medical injection system that uses the medicine injecting catheter 1 according to Embodiment 1. A medicine supply 23 and a pressure gage 22 are connected to the proximal end of the medicine injecting catheter 1 through a connector 21, and a controller 24 is connected to the pressure gage 22 and the medicine supply 23.

The pressure gage 22 detects the pressure in the medicine supply lumen 3 of the medicine injecting catheter 1. The medicine supply 23, which includes a syringe pump, for example, supplies a medicine to the medicine supply lumen 3 of the medicine injecting catheter 1. The controller 24 may control the supplying of the medicine from the medicine supply 23 so that the pressure detected by the pressure gage 22 in the medicine supply lumen 3 of the medicine injecting catheter 1 may be constant.

The medical injection system thus arranged is capable of holding the pressure in the medicine supply lumen 3 at a constant level.

An example of operation of the medicine injecting catheter 1 according to Embodiment 1 will be described below.

First, as depicted in FIG. 7, a guiding catheter G is inserted into the body of a patient, e.g., into the heart, and the medicine injecting catheter 1 is inserted into the guiding catheter 1 until the distal end of the medicine injecting catheter 1 projects out of the guiding catheter G.

The medicine supply lumen 3 of the medicine injecting catheter 1 has been filled with medicine by the medicine supply 23. When the cover 9 is not held in contact with an inner wall W of the heart, since no external forces act on the cover member 9, the cover 9 is positioned in the first position P1 so that the distal end of the injection needle is housed inside the cover member 9. As the distal end of the injection needle 8 is housed in the inner space 10 and the lumen 7 in the tubular body 6 is closed, no medicine leaks out of the injection needle 8.

The medicine injecting catheter 1 is advanced until the distal end of the cover member 9 is brought into contact with the inner wall W of the heart. When the medicine injecting catheter 1 pressed against the inner wall W of the heart, as depicted in FIG. 8, the cover member 9 is moved into the second position P2. The distal end of the injection needle 8 projects forwardly from the inner space 10 and pierces the inner wall W of the heart, and the lumen 7 in the tubular body 6 is opened. The pressure in the medicine supply lumen 3 is lowered, and based on a detection signal from the pressure gage 22 that has detected the pressure drop, the controller 24 actuates the medicine supply 23 to start supplying the medicine to the medicine supply lumen 3. The medicine in the medicine supply lumen 3 is injected into the heart tissue through the lumen 7 in the tubular body 6 and the injection needle 8.

When the inner wall W of the heart moves away from the medicine injecting catheter 1 and leaves (i.e., becomes separated from) the distal end of the cover member 9 as the heart pulsates, the injection needle 8 is pulled out of the inner wall W of the heart, and at the same time the cover member 9 moves from the second position P2 to the first position P1, whereupon the lumen 7 in the tubular body 6 is closed. Therefore, the medicine stops being injected from the injection needle 8.

When the inner wall W of the heart moves again toward the medicine injecting catheter 1 and the cover member 9 moves from the first position P1 to the second position P2 as the heart pulsates, the distal end of the injection needle 8 projects forwardly from the inner space 10 and pierces the inner wall W of the heart, whereupon the lumen 7 in the tubular body 6 is opened. The medicine is now once again injected into the heart tissue.

In this manner, the injection of the medicine into the heart tissue and the ceasing of the injection of the medicine are repeated as the heart pulsates.

When the cover member 9 is positioned in the first position P1, having the distal end of the injection needle 8 stored in the inner space 10, the diaphragm mechanism 15 closes the lumen 7 in the tubular body 6, and when the cover member 9 is positioned in the second position P2, having the distal end of the injection needle 8 projecting forwardly from the inner space 10, the diaphragm mechanism 15 opens the lumen 7 in the tubular body 6. Therefore, the injection needle 8 automatically pierces the inner wall W of the heart in synchronism with the pulsation of the heart for reliably injecting the medicine into the heart tissue without confirming whether the injection needle has pierced the inner wall W of the heart or not. When the injection needle 8 is pulled out of the inner wall W of the heart, the injection of the medicine from the injection needle 8 is automatically stopped to prevent the medicine from leaking out. Consequently, an appropriate amount of medicine can reliably be administered, and the medicine is prevented from being unnecessarily injected into regions other than the target region.

Because the injection of the medicine and the ceasing of the injection of the medicine are automatically carried out in response to the movement of the cover member 9 with respect to the catheter body 2, the injecting procedure is simplified.

The tissue into which the mediation is injected is not limited to heart tissue. Though the medicine injecting catheter is able to inject the medicine automatically into a heart by utilizing the pulsation of the heart, the medicine injecting catheter may not necessarily require a tissue to pulsate for injecting the medicine into the tissue. It is possible for the medicine injecting catheter to inject the medicine into other organs by pressing the distal end of the catheter against the body wall and to stop injecting the medicine by releasing the distal end of the

### Embodiment 2

In Embodiment 1, the opening and closing mechanism for opening and closing the lumen 7 in the tubular body 6 includes the diaphragm or diaphragm mechanism 15. However, the opening and closing mechanism is not limited to the diaphragm mechanism 15.

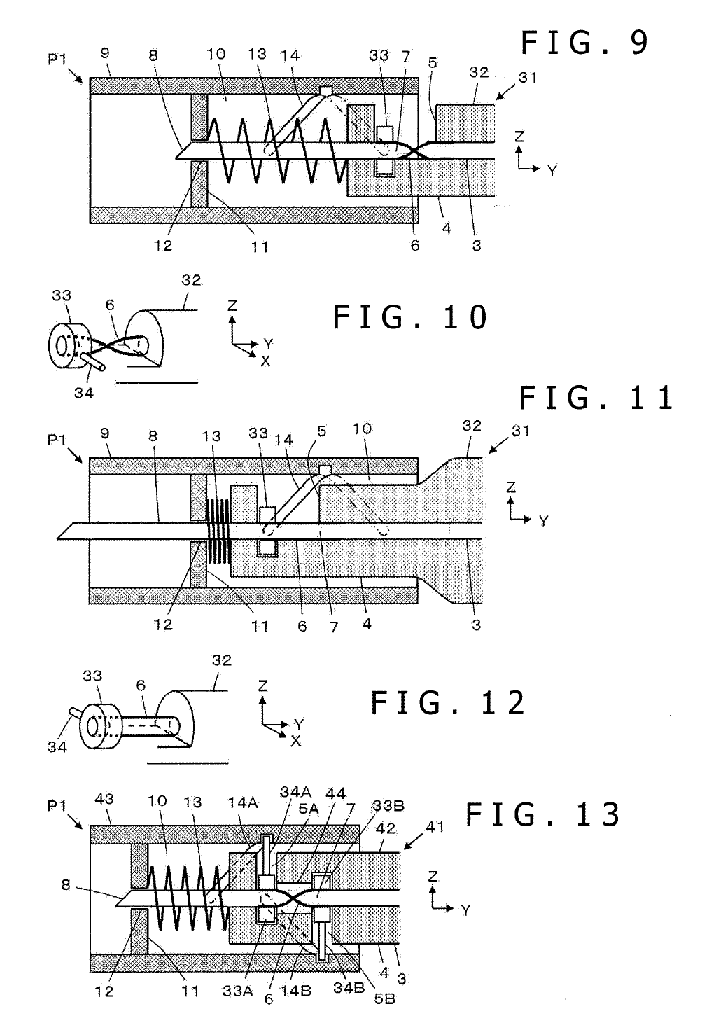

FIG. 9 depicts the makeup of a medicine injecting catheter 31 according to Embodiment 2. The medicine injecting catheter 31 includes an elongate catheter body 32 and a cover member 9 reciprocally movably disposed on the distal end of the catheter body 32.

In the catheter body 32, a disk-shaped rotor 33 is disposed in the recess 5 in the reduced-diameter portion 4, in place of the diaphragm mechanism 15 in the catheter body 2 used in the medicine injecting catheter 1 according to Embodiment 1. The rotor 33 serves as an opening and closing mechanism and is fixed to an outer circumferential surface of an end (an end in the −Y direction) of the tubular body 6. The tubular body 6 has an end rotatably coupled or connected to the medicine supply lumen 3 and another end (an end in the +Y direction) fixed to the medicine supply lumen 3.

The rotor 33 is disposed in the recess 5 in the reduced-diameter portion 4 for rotation about the central axis of the catheter body 32. As depicted in FIG. 10, an operating member 34 that extends radially outwardly is mounted on a side of the rotor 33. The operating member 34 has a distal end inserted or positioned in the operating member guide groove 14 defined in the inner wall surface of the cover member 9, so that the angular position of the operating member 34 varies depending on the relative position of the cover member 9 in the Y direction with respect to the catheter body 32, causing the tubular body 6 to twist upon rotation of the rotor 33.

When the cover member 9 is positioned in the first position P1 depicted in FIG. 9, the operating member 34 of the rotor 33 that is inserted in the operating member guide groove 14 of the cover member 9 is in an angular position oriented essentially in the +X direction as depicted in FIG. 10, in which the tubular body 6 in the form of a soft tube is twisted thereby to close the lumen 7 in the tubular body 6.

When the cover member 9 moves in the +Y direction with respect to the catheter body 32 until it is positioned in the second position P2 depicted in FIG. 11, since the operating member guide groove 14 of the cover member 9 extends helically, the operating member 34 of the rotor 33 that is inserted in the operating member guide groove 14 of the cover member 9 is brought into an angular position oriented essentially in the −X direction, untwisting the tubular body 6 thereby to open the lumen 7 in the tubular body 6, as depicted in FIG. 12.

According to Embodiment 2, therefore, when the cover member 9 is positioned in the first position P1, having the distal end of the injection needle 8 in the −Y direction stored in the inner space 10, the lumen 7 in the tubular body 6 is closed, and when the cover member 9 is positioned in the second position P2, having the distal end of the injection needle 8 in the −Y direction projecting forwardly from the inner space 10, the lumen 7 in the tubular body 6 is opened. Therefore, as with Embodiment 1, the injecting procedure is simplified, and at the same time the medicine can automatically and reliably be injected into the heart tissue in synchronism with the pulsation of the heart, and the medicine is prevented from leaking from the injection needle 8 when the injection needle 8 is pulled out of the inner wall W of the heart.

### Embodiment 3

In Embodiment 2, the single rotor 33 fixed to one end of the tubular body 6 serves as the opening and closing mechanism. However, respective rotors may be fixed to both ends of the tubular body 6, and may twist the tubular body 6 from both of its ends for closing the lumen 7 in the tubular body 6.

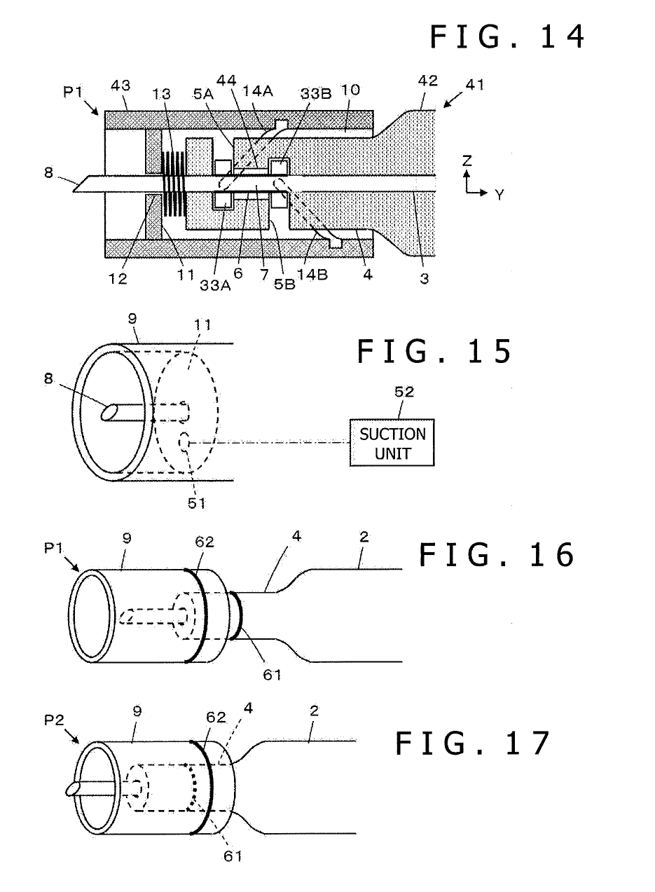

FIG. 13 depicts the makeup of a medicine injecting catheter 41 according to Embodiment 3. The medicine injecting catheter 41 includes an elongate catheter body 42 and a cover member 43 reciprocally movably disposed on the distal end of the catheter body 42.

The catheter body 42 has rotors 33A and 33B disposed in a pair of recesses 5A and 5B, respectively, that are defined in the reduced-diameter portion 4 at a spaced interval in the Y direction, in place of the single rotor 33 disposed in the recess 5 in the reduced-diameter portion 4 of the catheter body 32 used in the medicine injecting catheter 31 according to Embodiment 2. The pair of rotors 33A and 33B serve as an opening and closing mechanism and are fixed to respective outer circumferential surfaces of both ends of the tubular body 6. The two ends of the tubular body 6 are rotatably coupled or connected to the medicine supply lumen 3.

The rotors 33A and 33B each have the similar structure as the rotor 33 according to Embodiment 2, and have respective operating members 34A and 34B extending radially outwardly from sides thereof.

Of the pair of recesses 5A and 5B, the recess 5A is defined in the +Z direction that extends from the central axis of the catheter body 42, and the other recess 5B is defined in the −Z direction (i.e., the negative Z direction or the downward Z direction in FIGS. 1 and 4 for example) that extends perpendicularly from the central axis of the catheter body 42. The recesses 5A and 5B are held in fluid communication with each other through a through hole 44 defined in the catheter body 42 on its central axis. The tubular body 6 extends in the through hole 44 and have both ends positioned in the respective recesses 5A and 5B and coupled or connected to the corresponding rotors 33A and 33B.

The cover member 43 has a pair of operating member guide grooves 14A and 14B defined in an inner wall surface thereof. The operating member guide groove 14A is disposed only in an inner wall surface of the cover member 43 that lies in the +Z direction, of the entire inner wall surface of the cover member 43, in agreement with the direction along which the recess 5A is defined in the catheter body 42. The operating member guide groove 14A being disposed only in the +Z direction is depicted in FIGS. 1 and 4 for example. The operating member guide groove 14A extends helically around the central axis of the cover member 43 along the Y direction. The other operating member guide groove 14B is disposed only in an inner wall surface of the cover member 43 that lies in the −Z direction, of the entire inner wall surface of the cover member 43, in agreement with the direction along which the recess 5B is defined in the catheter body 42. The operating member guide groove 14B extends helically around the central axis of the cover member 43 along the Y direction, wound in a twisting direction opposite the operating member guide groove 14A.

The operating member 34A that extends radially outwardly from the side of the rotor 33A has a distal end inserted in the operating member guide groove 14A of the cover member 43, whereas the operating member 34B that extends radially outwardly from the side of the rotor 33B has a distal end inserted in the operating member guide groove 14B of the cover member 43. Consequently, the angular positions of the respective operating members 34A and 34B vary depending on the relative position of the cover member 43 with respect to the catheter body 42 in the Y direction. As the rotors 33A and 33B are rotated in opposite directions, the tubular body 6 is twisted from both ends of the tubular body 6.

When the cover member 43 is positioned in the first position P1 depicted in FIG. 13, the operating member 34A of the rotor 33A that is inserted in the operating member guide groove 14A of the cover member 43 is in an angular position oriented essentially in the +Z direction, and the operating member 34B of the rotor 33B that is inserted in the operating member guide groove 14B of the cover member 43 is in an angular position oriented essentially in the −Z direction, as depicted in FIG. 13. At this time, the tubular body 6 which in the form of a soft tube is twisted from both ends of the tubular body 6 in the through hole 44 thereby closing the lumen 7 in the tubular body 6.

When the cover member 43 moves in the +Y direction until it is positioned in the second position P2 depicted in FIG. 14 with respect to the catheter body 42, the operating member 34A of the rotor 33A that is inserted in the operating member guide groove 14A of the cover member 43 and the operating member 34B of the rotor 33B that is inserted in the operating member guide groove 14B of the cover member 43 are brought into respective angular positions both oriented essentially in the −X direction, untwisting the tubular body 6 thereby to open the lumen 7 in the tubular body 6.

According to Embodiment 3, therefore, when the cover member 43 is positioned in the first position P1 in which the distal end of the injection needle 8 in the −Y direction is stored or housed in the inner space 10, the lumen 7 in the tubular body 6 is closed, and when the cover member 43 is positioned in the second position P2 in which the distal end of the injection needle 8 in the −Y direction -projects forwardly or in the distal direction from the inner space 10, the lumen 7 in the tubular body 6 is opened. Therefore, as with Embodiments 1 and 2, the injecting procedure is simplified, and at the same time the medicine can automatically and reliably be injected into the heart tissue in synchronism with the pulsation of the heart, and the medicine is prevented from leaking from the injection needle 8 when the injection needle 8 is pulled out of the inner wall W of the heart.

According to Embodiment 3, furthermore, because the pair of rotors 33A and 33B rotate in mutually opposite directions upon movement of the cover member 43 with respect to the catheter body 42, when the rotors 33A and 33B rotate through an angle that is ½ of the angle through which the rotor 33 according to Embodiment 2 rotates, the tubular body 6 is twisted through the same angle. In other words, the distance between the first position P1 and the second position P2 in the Y direction may be smaller, making it possible to reduce the size of the cover member 43.

### Embodiment 4

In Embodiment 1 described above, the medicine injecting catheter may further include a capture mechanism or trap mechanism for capturing (e.g., drawing-in) the inner wall W of the heart into the front end portion of the cover member 9 by evacuating the inner space 10 in the cover member 9.

As depicted in FIG. 15, the capture mechanism includes a suction hole 51 defined in the partition plate 11 of the cover member 9 and a suction unit 52 connected to the suction hole 51. For example, the suction unit 52 may be connected to the connector 21 of the medicine injecting system depicted in FIG. 6, the catheter body 2 of the medicine injecting catheter 1 may have a suction lumen defined therein independently of the medicine supply lumen 3, and the suction unit 52 may be connected to the suction hole 51 in the cover member 9 through the suction lumen and the connector 21.

When the suction unit 52 draws air from the suction lumen to evacuate the inner space 10 in the cover member 9, the inner wall W of the heart is captured or held in the front end portion of the cover member 9. It is thus possible to inject the medicine more reliably into the heart tissue.

The capture mechanism may similarly be incorporated into the medical injecting catheter 31 according to Embodiment 2 and the medical injecting catheter 41 according to Embodiment 3.

### Embodiment 5

In Embodiment 1 described above, contrast markers 61 and 62 may be provided on the outer circumferential surface of the reduced-diameter of the catheter body 2 and the outer circumferential surface of the cover member 9, respectively. An example of such contrast markers 61, 62 is shown in FIG. 16. These contrast markers 61 and 62 serve to assist in grasping or identifying the positions of the reduced-diameter of the catheter body 2 and the cover member 9 by contrast X-rays when the medicine injecting catheter 1 is inserted into the body of a patient. The contrast markers 61 and 62 may be made of a material easily recognizable by contrast X-rays, e.g., gold, platinum, iridium, tungsten, or an alloy of any of them, or a silver-palladium alloy, or the like.

By identifying the position of the reduced-diameter of the catheter body 2 and the position of the cover member 9 using the contrast markers 61 and 62, it is possible to decide on an X-ray image whether the cover member 9 is positioned in the first position P1 as depicted in FIG. 16 or positioned in the second position P2 as depicted in FIG. 17.

Contrast markers may similarly be provided on both the reduced-diameter portion of the catheter body and the cover member of the medicine injecting catheter 31 according to Embodiment 2, the medicine injecting catheter 41 according to Embodiment 3, and the medicine injecting catheter according to Embodiment 4.

In Embodiments 1 through 5, the medicine into the inner wall W. However, the injection needle 8 may similarly pierce the outer wall of the heart and inject the medicine into the outer wall. The object to be treated is not limited to the heart, but the medicine injecting catheter according to the present invention is applicable where a medicine is to be injected into living bodies through various body walls thereof, such as inner and outer walls of other organs, etc.

The medicine may be any optional therapeutic substances. The therapeutic effects may include cardiac wall strengthening, scaffolding, proangiogenesis, cell replenishment, and tissue repair or regeneration by way of apoptosis and necrosis prevention. The therapeutic substances include a biocompatible mono- or multi-component material that can be injected, beads on a polymer base, and polymer hydrogels. Other therapeutic substances include a fibrin adhesive, collagen, alginate, a synthetic polymeric material such as polyethylene glycol or the like, and chitosan, etc. Though the therapeutic substances may include the above materials only, they are not limited thereto. For example, they may include, individually or in any optional combination, stem cells such as induced pluripotent stem (iPS) cells or the like, fibroblast, cells such as skeletal cells or the like, protein, plasmid, genes, growth factors, chemoattractant, synthetic polypeptide, various pharmaceutical compositions, and other therapeutically useful substances.

The detailed description above describes embodiments of a medicine injecting catheter and medicine injecting system representing examples of the inventive medicine injecting catheter and medicine injecting system disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medicine injecting catheter for injecting medicine into a wall of a living body, the medicine injecting catheter comprising:
an elongate catheter body extending in an axial direction, the catheter body possessing a distal end and including a medicine supply lumen extending in the catheter body;
a tubular body connected to the distal end of the catheter body, the tubular body including a lumen in fluid communication with the medicine supply lumen;
an injection needle connected to the distal end of the tubular body, the injection needle possessing a distal end;
a hollow cover member that includes an inner space in which is accommodated at least the tubular body, the cover member being disposed on a distal end portion of the catheter body for reciprocating movement along the axial direction of the catheter body between a first position in which the distal end of the injection needle is housed in the inner space and a second position in which the distal end of the injection needle projects forwardly from the inner space;
an opening and closing mechanism that includes an operating member rotatable in opposite circumferential directions of the catheter body in the inner space in the cover member, for opening and closing the lumen in the tubular body depending on an angular position of the operating member;
a motion converting mechanism for converting the reciprocating movement of the cover member relative to the catheter body into rotary movement of the operating member to cause the opening and closing mechanism to close the lumen in the tubular body when the cover member is positioned in the first position and to cause the opening and closing mechanism to open the lumen in the tubular body when the cover member is positioned in the second position; and
a resilient member which is disposed between the catheter body and the cover member and which resiliently presses the cover member forwardly of the catheter body toward the first position.

2. The medicine injecting catheter according to claim 1, wherein
the tubular member is pliable; and
the opening and closing mechanism includes a diaphragm mechanism for changing an opening area of the lumen in the tubular body depending on the angular position of the operating member.

3. The medicine injecting catheter according to claim 1, wherein
the tubular member is pliable; and
the opening and closing mechanism includes at least one rotor for twisting the tubular body depending on the angular position of the operating member.

4. The medicine injecting catheter according to claim 1, wherein
the motion converting mechanism includes an operating member guide groove defined helically in an inner circumferential surface of the cover member around a central axis of the cover member; and
the operating member includes a radially extending projection that is positioned in and movable along the operating member guide groove.

5. The medicine injecting catheter according to claim 1, wherein
the cover member includes a partition plate fixed to the cover member and disposed in the inner space of the cover member perpendicular to the axial direction, the partition plate including g a through hole through which the injection needle extends; and
the resilient member is disposed between a front end of the catheter body and the partition plate of the cover member.

6. The medicine injecting catheter according to claim 1, further comprising a capture mechanism for capturing a portion of the wall of the living body in a front end portion of the cover member by evacuating the inner space in the cover member.

7. The medicine injecting catheter according to claim 1, wherein the catheter body and the cover member each include a respective contrast marker.

8. A medicine injecting system comprising:
the medicine injecting catheter according to claim 1;
a pressure gage that detects a pressure in the medicine supply lumen of the medicine injecting catheter;
a medicine supply that supplies a medicine to the medicine supply lumen of the medicine injecting catheter; and
a controller that controls supply of the medicine from the medicine supply to the medicine supply lumen so that the pressure detected by the pressure gage in the medicine supply lumen of the medicine injecting catheter is constant.

9. A medicine injecting catheter for injecting medicine into a wall of a living body, the medicine injecting catheter comprising:
an elongate catheter body extending in an axial direction, the catheter body possessing a distal end and including a medicine supply lumen extending in the axial direction inside the catheter body and opening to the distal end of the catheter body;
a tubular body connected to the distal end of the catheter body, the tubular body including a lumen and being made of compressible material that is compressed upon application of a force to an outer periphery of the tubular body to close the lumen in the tubular body;
an injection needle connected to the tubular body, the injection needle possessing a distal end and including a lumen that opens to the distal end of the injection needle;
the catheter body, the tubular body and the injection needle being movable together as a unit;
a cover member surrounding an interior in which is located at least a portion of the tubular body, the cover member and the catheter body being relatively movable between a first position in which a distal-most end of the injection needle is proximal of a distal-most end of the cover member so that the distal-most end of the injection needle is covered by the cover member and a second position in which the distal-most end of the injection needle protrudes distally beyond the distal-most end of the cover member so that the distal-most end of the injection needle is outside the tubular cover to puncture the living body;
an opening and closing mechanism for closing the lumen in the tubular body and opening the lumen in the tubular body, the opening and closing mechanism comprising at least one tubular body contacting part movable between one position in which the tubular body contacting part contacts the tubular body and applies a compressive force to the tubular body that closes the lumen in the tubular body and an other position in which the tubular body contacting part no longer applies the compressive force to the tubular body so that the lumen in the tubular body is open;
the opening and closing mechanism also comprising an operating member that is operatively connected to the tubular body contacting part and the cover member so that when the cover member is in the first position with the distal-most end of the injection needle covered by the cover member, the tubular body contacting part is in the one position to close the lumen in the tubular body and prevent medicine from flowing into the injection needle, and when the cover member is shifted to the second position with the distal-most end of the injection needle protruding distally beyond the distal-most end of the cover member, the tubular body contacting part is moved to the other position so that the lumen in the tubular body is open to permit medicine to flow into the injection needle.

10. The medicine injecting catheter according to claim 9, wherein the tubular body contacting part comprises at least one blade mounted in a frame.

11. The medicine injecting catheter according to claim 9, wherein the tubular body contacting part comprises a plurality of blades and the operation member projects radially outwardly from the frame and engages a groove formed in an inner surface of the cover member.

12. The medicine injecting catheter according to claim 9, further comprising a spring that applies a biasing force between the cover member and the catheter

13. The medicine injecting catheter according to claim 9, wherein the tubular body contacting part comprises at least one blade mounted in a frame, the operation member is a projection that projects outwardly away from the frame and engages a helical groove in an inner surface of the cover member so that the movement of the cover member from the first position to the second position causes the projection to move in the helical groove.

14. The medicine injecting catheter according to claim 9, further comprising a partition plate positioned in the interior of the cover member and fixed to the cover member, and a resilient member positioned between the partition plate and the catheter body to urge the cover member in the first position.

15. The medicine injecting catheter according to claim 9, further comprising a partition plate positioned in the interior of the cover member and fixed to the cover member, and a resilient member positioned between the partition plate and the catheter body that urges the cover member toward the first position.

16. The medicine injecting catheter according to claim 15, further comprising a through hole in the partition plate, the injection needle being located in the through hole in the partition plate and being movable relative to the partition plate.

17. The medicine injecting catheter according to claim 9, further comprising a partition plate positioned in the interior of the cover member to divide the interior of the cover member into one area distal of the partition plate and an other area proximal of the partition plate, and a suction hole passing through the partition plate and connectable to the suction hole to draw a suction in the first area in the interior of the cover member.

18. The medicine injecting catheter according to claim 9, wherein the catheter body and the cover member each include a respective contrast marker.

19. A method comprising:
inserting a catheter body into a patient's body, the catheter body possessing a distal end, the catheter body including a medicine supply lumen extending in the catheter body and opening at the distal end of the catheter body, the catheter body being connected to a tubular body which includes a lumen in communication with the medicine supply lumen, the tubular body being connected to an injection needle which possesses a distal end, the catheter body and the injection needle being movable together, at least a portion of the tubular body being positioned in an interior of a cover member, and a rotatable operation member positioned inside the cover member;
moving the cover member and the catheter body in a forward direction toward a wall inside the patient's body while a compressive force is applied to the tubular body to close the lumen in the tubular body to completely block flow of medicine through the lumen in the tubular body;
moving the cover member in the forward direction so that a distal end of the cover member contacts the wall inside the patient's body to stop the movement of the cover member in the forward direction, and moving the catheter body in the forward direction relative to the cover member after the distal end of the cover member contacts the wall inside the patient's body so that the distal end of the injection needle extends distally beyond a distal end of the cover member and punctures the wall of the patient's body;
the moving of the catheter body in the forward direction relative to the cover member after the distal end of the cover member contacts the wall inside the patient's body causing the operation member to rotate, and the rotation of the operation member automatically removing the compressive force applied to the tubular body to open the lumen in the tubular body; and
following the opening of the lumen in the tubular body, the medicine flowing through the medicine supply lumen, through the lumen in the tubular body and through the injection needle so that the medicine exits the injection needle by way of the distal end of the injection needle and is introduced into tissue in the wall of the patient's body.

20. The method according to claim 19, wherein the patient's body includes a heart, and the wall of the patient's body is an inner wall of the patient's heart.
